(19) United States
(12) Patent Application Publication
(10) Pub. No.: US 2024/0424001 A1
(43) Pub. Date: Dec. 26, 2024

(54) LOCAL DELIVERY OF GROWTH AND REPAIR PROMOTING COMPOUNDS FOR TREATING, REDUCING AND/OR PREVENTING STRESS URINARY INCONTINENCE AND FECAL INCONTINENCE

(71) Applicant: STATERA PHARMA INC., GLADSTONE, NJ (US)

(72) Inventors: EBRAHIM VERSI, GLADSTONE, NJ (US); NICHOLAS SIDDLE, TATTINGSTONE, IPSWICH (GB)

(73) Assignee: STATERA PHARMA INC., GLADSTONE, NJ (US)

(21) Appl. No.: 18/690,279

(22) PCT Filed: Sep. 9, 2022
(86) PCT No.: PCT/US2022/076156

§ 371 Date: Mar. 8, 2024

Related U.S. Application Data
(60) Provisional application No. 63/242,566, filed on Sep. 10, 2021.

(51) Int. Cl. A61K 31/5685; A61K 9/00; A61K 38/25

(52) U.S. Cl. CPC A61K 31/5685; A61K 9/0034; A61K 38/25

(57) ABSTRACT

The present invention relates to compositions and local delivery methods to increase pelvic tissue integrity and the treatment, reduction and/or prevention of stress urinary incontinence [SUI] and fecal incontinence [FI] due to weakness in pelvic floor. The treatment comprises of growth and/or repair promoting compound(s) [GRPCs] including but not limited to androgens, androgen receptor modulators, ghrelin, a ghrelin analog, an estrogen, an estrogen receptor modulator, or a combination thereof for delivery into the vagina, anorectal canal or through the mucosa in these viscera to treat pelvic floor disorders.

### CROSS REFERENCE TO RELATED APPLICATION

This application claims priority to U.S. Provisional Patent Application No. 63/242,566, filed on Sep. 10, 2021, the contents of which are hereby incorporated by reference herein for all purposes.

### BACKGROUND OF THE INVENTION

### Technical Field

The present invention relates to compositions and local delivery methods to increase pelvic tissue integrity and the treatment, reduction and/or prevention of stress urinary incontinence [SUI] and fecal incontinence [FI] due to weakness or damage to pelvic floor and associated structures. The treatment comprises of growth and/or repair promoting compound(s) [GRPCs] including but not limited to androgens, androgen receptor modulators, ghrelin, a ghrelin analog, an estrogen, an estrogen receptor modulator, or a combination thereof for delivery into the vagina, anorectal canal or through the mucosa in these viscera to treat pelvic floor disorders.

### Related Art

Stress urinary incontinence [SUI] and fecal incontinence [FI] occurs during activities such as coughing or sneezing which cause an increase in intraabdominal pressure. They result from failure of one, or more frequently, several of the multiple control mechanisms that normally ensure continence of urine or feces during such increase in intraabdominal pressure termed a “stress” activity. These control mechanisms involve structural anatomic features, intact neurological mechanisms, a fully functional local vasculature, anatomical integrity of all of the component tissues in the region of the urethra and bladder neck and anal canal. These all act in concert as the ‘functional’ urethral sphincter or anal sphincter. (1)

The pathogenesis of SUI/FI involves both traumatic damage to any of the continence control mechanisms during childbirth and reduced tissue integrity caused severally by postmenopausal hormone deficiency and other complex parts of the aging process including but not limited to declining growth hormone secretion.

Urinary continence is maintained at the level of the bladder neck (2, 3) but when this fails, continence is still maintained by components of the urethra including the rhabdosphincter and the pelvic floor (levator) musculature and controlling nerve supply. In addition, the vascularity, collagen and connective tissues and urethra mucosal coaptation all also contribute to the continence mechanisms. Broadly speaking, the urethral deficiency can be categorized as urethral hypermobility or intrinsic sphincter deficiency. In practice patients present with both, to varying degrees of severity. (2, 3, 4).

Currently, common treatments of SUI include lifestyle changes, pelvic floor exercises (Kegels), pharmacological treatment to augment sphincter tone, injection of bulking agents to augment closure of bladder neck, injection of stem cells into the rhabdosphincter of the urethra and a variety of continence surgeries. (4) These surgical interventions attempt to compensate for, or restore, defects in the supports and positioning of pelvic anatomy or to enhance ineffective bladder or anal sphincter closure. Surgical Interventions include procedures designed to elevate the bladder neck and proximal urethra or to stabilize the urethra during increases in intraabdominal pressure, sphincteroplasty and correction of co-existing conditions such as pelvic floor relaxation.

The mechanisms resulting in fecal incontinence are similar to those resulting in urinary incontinence. The internal and external anal sphincter and the levator ani all play a role but in addition, the physical character of the stool is also important.

For FI surgical interventions also attempt to restore defects in the supports and positioning of relevant pelvic anatomy or to enhance ineffective anal sphincter closure. Therapeutic approaches include sling procedures, sphincteroplasty, artificial sphincters, neuromodulation and correction of co-existing conditions such as rectal prolapse. (5, 6)

Estrogen deficiency is common to all women after menopause because estrogen availability typically falls by 90% once the menopausal transition is complete. However, this is not the only significant hormonal decline with age. It is often overlooked that female androgen levels also fall significantly. Following menopause, the source of androgens is still, to some extent, the ovary (7) and the adrenal gland which produces both androstenedione (A4) and dehydroepiandrosterone (DHEA) that can be a precursor of other more potent androgens. (8) However, ovarian output of testosterone declines with age and adrenal output begins to decline in women even before menopause, from the mid-twenties. Consequently, for many post-menopausal women androgen levels are very low. Growth hormone and related GRPCs also fall with age and could be a part of the negative impact of aging on continence mechanisms. (9)

The prevalence of urinary incontinence and of other lower urinary tract symptoms increases after the menopause and affects between 38% and 55% of women aged over 60 years. (10, 11) In community dwelling women, the reported prevalence of FI varies widely from 2.2% to 24% and rises with advancing age. (12)

Therefore, it is possible that pelvic floor dysfunction is due to both the aging process and post-menopausal hormone deficiency, which the present inventors theorize is a result of declining activity of the estrogenic, androgenic and growth hormone axes.

Administration of estrogens to restore the loss of structural tissue integrity and function of the vagina following the menopause is an established intervention. This approach successfully reverses estrogen deficiency related vaginal epithelial changes but the effects on SUI are mixed and sometimes deleterious. (13) Additionally, some epidemiological studies suggest a deleterious effect of estrogens on FI in some women. (12) Thus, current evidence of drug treatment efficacy for FI is not consistent as there are few good quality studies and there is no agreed consensus on medical management of FI

As an example, estrogen treatment may be given orally with good effect on the pelvic tissues, but this predicates impact on the body generally which may not be desired for a variety of reasons, not least of which is expressed concerns, whether evidence based or not, related to cancer and cardiovascular disease.

In light of the shortcomings of the prior art, the present invention provides for new and inventive methods and compositions for treating and/or preventing SUI and/or FI.

### SUMMARY OF THE INVENTION

To overcome the shortcomings of previous treatment for SUI and/or FI, the present invention provides treatment and methods to increase pelvic tissue integrity by local delivery of growth and/or repair promoting compound(s) [GRPCs] to areas having contact with the pelvic floor of a treatable subject. Also, that appropriate stimulation of tissue growth and repair alone or in combination with pelvic floor muscle exercises might obviate the need for surgery.

In one aspect, the present invention provides for a method of local delivery of at least one therapeutic compound to treat pelvic floor disorders in a human subject, wherein the therapeutic compound is selected from the group consisting of an active form of ghrelin, a ghrelin analog, an androgen, an estrogen, a selective estrogen receptor modulator, an androgen receptor modulator or a combination thereof and wherein the pelvic floor disorder is selected from the group consisting of fecal incontinence, stress urinary incontinence, pelvic organ prolapse, rectal prolapse, and combination thereof. A preferred combination comprises both an androgen such as dehydroepiandrosterone sulphate (DHEAS), dehydroepiandrosterone (DHEA), androstenedione (A), testosterone (T), and dihydrotestosterone and an estrogen including but not limited to estrone, estradiol, or estriol.

Local delivery of at least one therapeutic compound to treat pelvic floor disorders in a human subject includes but is not limited to insertion into the vagina, into or through the vagina wall, into and/or through the anal canal wall, into the rectum, into and/or through the rectal wall, through the perineum and the bladder wall.

The present invention provides for local delivery of at least one therapeutic compound as disclosed above to treat pelvic floor disorders in a female human subject, wherein the pelvic floor disorder is selected from the group consisting of uterovaginal prolapse, vaginal vault prolapse, an anterior vaginal wall prolapse, cystocele, a posterior vaginal wall prolapse and rectocele.

In yet another aspect, the present invention provide for use of at least one growth and/or repair promoting compound (GRPC) as a therapeutic compound in a medicament for the treatment of pelvic floor disorders in a human subject, wherein the pelvic floor disorder is selected from the group consisting of fecal incontinence, stress urinary incontinence, pelvic organ prolapse, rectal prolapse and a combination thereof, and wherein the at least one therapeutic compound is selected from the group consisting of an active form of ghrelin, a ghrelin analog, an androgen, an estrogen, a selective estrogen receptor modulator, an androgen receptor modulator or a combination thereof.

The term GRPC includes sex hormones and growth factors such as, but not limited to, growth hormone secretagogues, analogues or mimetics that exert a positive effect on pelvic tissue. The need is localized to the pelvic floor tissues and systemic exposure in some cases may not be desirable, so a ‘local’ therapy is desirable. Importantly, the present invention overcomes the potential negative effects of systemic exposure and may allow higher exposure of the therapeutic agent to the target tissues by the use of local therapy as locally absorbed hormones reach the pelvic tissues before they enter the general circulation. Consequently, plasma concentrations will be lower than pelvic tissue concentrations.

The delivery system of the present invention allows for optimum transfer of GRPCs from vehicle or device to target tissues with maximum patient convenience and minimal systemic exposure and hence impact in terms of side effect. This delivery could be into the vagina or ano-rectal canal and absorption would take place through the vaginal or ano-rectal mucosa or by injection into the pelvic tissues. For trans-mucosal treatment, the delivery system may be a vaginal ring, a vaginal pessary, gel, cream, ointment, suppository, or a microarray needle system.

In another aspect, the present invention provides for a composition comprising at least one therapeutic compound selected from the group consisting of an active form of ghrelin, a ghrelin analog, an androgen, an estrogen, a selective estrogen receptor modulator, an androgen receptor modulator or a combination thereof. Preferably the composition comprises at least an estrogen and androgen. Additionally, the composition may comprise a ghrelin or analog thereof. The composition may include a pharmaceutically acceptable carrier.

The amount of active therapeutic compounds present in the composition depends on the strength of the final composition. In one embodiment, the at least one therapeutic compound is present in amounts ranging from about 0.2 mg per dose to about 200 mg per dose, preferably from about 1 mg to about 30 mg per dose, preferably from about 2 mg to about 20 mg per dose, more preferably from about 10 mg to about 20 mg per dose. In particular suppository dosage forms, the at least one therapeutic compound is present in amounts from about 1 mg to about 30 mg per dose, preferably from about 10 mg to about 20 mg per dose. In particular cream or gel dosage forms, the at least one therapeutic compound is present in amounts from about 1 mg to about 50 mg per dose, preferably from about 10 mg to about 30 mg per dose.

The composition may be administered daily, twice a week or weekly depending on administration mode and for at least 3 months, preferably for at least 12 months.

The compositions of the present invention and the important local delivery has a high local effect while reducing systemic side effects due to the lower serum concentrations and less systemic side effects when compared to an oral route of administration thus making it local delivery to pelvic floor area as an ideal route of administration.

These and other aspects of the invention are discussed more in the detailed description below.

### DETAILED DESCRIPTION OF THE INVENTION

The present intervention teaches the use of local administration of GRPCs in the treatment of SUI and FI. GRPCs may be administered as monotherapy or combination therapy, as appropriate, to ensure either an optimum sex hormone [estrogen/androgen] milieu for tissue growth and repair or to add to that an additional growth factor, a form of ghrelin or a ghrelin analog that binds the ghrelin receptor system locally so that the total GRPC combination optimally stimulates growth and or repair to the pelvic tissues, reversing menopausal and/or aging changes. The objective is to improve tissue structure and function in all the pelvic floor tissues, specifically the levator ani muscles, the periurethral tissues and bladder neck area and all structures that contribute to urinary continence or fecal continence and to prevent or treat SUI or FI.

The terms used in this specification generally have their ordinary meanings in the art, within the context of this invention and in the specific context where each term is used. Certain terms are defined below to provide additional guidance in describing the compositions and methods of the invention and how to make use of them.

### Definitions

The term “estrogen receptor” refers to any protein in the nuclear receptor gene family that binds to estrogen. Human estrogen receptor in the present invention includes the alpha-receptor isoform (referred to herein as “ER-alpha”) in addition to any additional isoforms as recognized by those of skill in the biochemistry arts.

The term “selective estrogen receptor modulator” (or “SERM”) is a compound that exhibits activity as an agonist or antagonist of an estrogen receptor (e.g., ER-alpha) in a tissue dependent manner. Thus, as will be apparent to those of skill in the biochemistry arts, compounds of the invention that function as SERMs can act as estrogen receptor agonists in some tissues (e.g., bone, vagina, bladder and urethra) and as antagonists in other tissue types such as breast.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively. “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless specified otherwise, all values provided herein can be assumed to include the term about.

The phrase “pharmaceutically acceptable” refers to molecular entities and compositions that are “generally regarded as safe” (GRAS), e.g., that are physiologically tolerable and do not typically produce an allergic or similar untoward reaction, such as gastric upset, dizziness and the like, when administered to an animal. Preferably, as used herein, the term “pharmaceutically acceptable” means approved by a regulatory agency of the Federal or state government or listed in the U.S. Pharmacopeia or other generally recognized pharmacopeia for the use in animals.

The term “carrier” refers to a diluent, adjuvant, excipient, or vehicle with which the compound is administered. Such pharmaceutical carriers can be sterile liquids, due to its high in solubility in water, oils, including those of petroleum, animal, vegetable or synthetic origin, such as peanut oil, soybean oil, mineral oil, sesame oil and the like. Carriers such as micelles or dextran can be used to deliver the agent in an aqueous solution or suspension. E. W. Martin describes suitable pharmaceutical carriers in “Remington's Pharmaceutical Sciences”.

The term “amount” as used herein refers to quantity or to concentration as appropriate to the context. The effective amount of a drug that constitutes a therapeutically effective amount varies according to factors such as the potency of a particular drug, the route of administration of the formulation, and to the mechanical system used to administer the formulation. A therapeutically effective amount of a particular drug can be selected by those of ordinary skill in the art with due consideration of such factors.

The delivery of localized therapy of the present invention largely confines the therapeutic impact to local tissues with higher exposures than could be achieved by systemic therapy, and this can be achieved by administration through the vagina and/or anorectal mucosa. Such an approach also allows for lower overall doses to be effective: the approach is in keeping with best medical practice. This targeted approach is potentially safer as non-pelvic tissues will have no or limited exposure to the treatment.

Androgens can have an influence on muscle, vasculature, collagen metabolism and neural function and hence could positively impact tissue integrity in the bladder neck and rectal/perianal area but it is not current practice to use androgen treatment as part of the armamentarium for treating SUI or FI.

The effects of ‘aging’ on continence may be mediated in part via reduced growth hormone availability, locally. Ghrelin, a growth hormone secretagogue, is better known for its effect on appetite, however it can have a role in maintaining tissue integrity which may also apply to pelvic floor tissues.

This present invention teaches that substances that improve the integrity of pelvic floor tissues would be used as therapeutics for the treatment of pelvic floor disorders. Pelvic floor disorders comprise of incontinence (urinary and/or fecal) and/or pelvic organ prolapse (ureterovaginal and/or rectal). Such therapeutic substances can be classified broadly as growth and/or repair promoting compound(s) [GRPCs] and comprise various agents such as sex hormones, sex hormone receptor modulators and growth factors such as, but not limited to, growth hormone secretagogues, analogs or mimetics that exert a positive effect on pelvic tissue. These agents could be effective as monotherapy noting that the administration of multiple therapeutic compounds with different mechanisms of actions, combination therapy will show both additive and/or synergistic effects.

In one broad aspect of this invention a potent androgen is used either alone or in combination with a potent estrogen with or without the addition of an active form of ghrelin e.g. n-octanoyl ghrelin [NAG] or des-acyl ghrelin [DAG] or an analog with the aim of optimizing pelvic tissue structure and function in order to treat pelvic floor dysfunction by improving tissue integrity.

The potent androgen could be dihydrotestosterone [DHT] and the potent estrogen could be estradiol [E2]. In women. E2 is the principal circulating estrogen before the menopause and the most potent of the estrogens so this is the logical candidate for treatment of estrogen deficiency changes.

The choice of DHT is based on potency, receptor binding activity and the question of aromatization of testosterone to estradiol. The androgen group consists of four hormones: dihydrotestosterone (DHT), testosterone, androstenedione and dehydroepiandrosterone. The relative potencies of these androgens are roughly 300%, 100%, 10% and 5% respectively; these relative potencies are approximate due to variations in species studied and the assay methodology used. (14)

The principal circulating androgen is testosterone. However, once it has been absorbed into certain target tissues, testosterone is converted to dihydrotestosterone [DHT] by the enzyme 5-alphareductase and in those tissues androgen action is mediated via DHT, preferentially. The androgen receptor [AR] binds both testosterone and DHT but the potency of DHT is greater than that of testosterone in part because the affinity of the AR for DHT is approximately 4-fold higher than for testosterone. (15, 16) Both androgens exert genomic [via the receptor mechanism] and non-genomic effects [via other mechanisms]. (17) In tissues where DHT is the relevant androgen and 5-alphareductase activity is low, the consequence is that testosterone has limited activity and administration of DHT rather than testosterone ensures maximum impact.

Ghrelin and its analogs are thought to promote tissue repair and growth and hence can be classified as GRPCs. The concept of using GRPCs for SUI and FI relies on the well accepted understanding that reduced function of the pelvic tissues and structures that maintain urinary and fecal continence follows from the consequences of the triad of childbirth trauma to the pelvis supporting structures, aging [with loss of growth hormone effects] andropause and menopause [with consequent severe deprivation of sex hormones]. Treatment through each of these three mechanisms could have a therapeutic effect, and with such combination, can show an additive or a synergistic effect.

The rationale for local treatment is that the agents target the desired tissues and not the body as a whole. This might require lower doses than required for systemic administration and should limit side effects that as re the result of off-target effects. Appropriate delivery vehicles that facilitate transvaginal or trans anal or transrectal therapeutic delivery can be used for such treatment.

Whereas conventional treatment of urinary incontinence and to a degree, fecal incontinence (18) has embraced local estrogen treatment even though the results are mixed. However, the potential for androgen supplementation has hitherto not been used or even considered as therapy. Currently, no human studies for the use of ghrelin for treating any pelvic floor disorders including incontinence has been found or noted by the inventors.

Surgical reconstruction for SUI or FI depends for its success on the presence of adequate tissue and adequate tissue strength for support. It is postulated that the treatments described will result improved tissue integrity and hence enhance surgical outcomes. In addition, the use of GRPCs through its improvement in pelvic tissue integrity including pelvic floor musculature such as the levator ani and urethral and anal sphincters may make a vital contribution to non-surgical treatments such as pelvic floor muscle exercises.

The method of local application that is most successful may vary from patient to patient-acceptability of the method of drug delivery is likely to be critical for treatment compliance. Daily administration of an estrogen preparation that absorbs via the vaginal mucosa/epithelium is a preferrable method of delivery. The vagina is intimately associated with the urethra and bladder neck thus vaginal delivery could also affect the lower urinary tract. However, it may be necessary or preferable in some women to deliver the therapeutic through the ano-rectal canal as would be the case in men.

Formulations for rectal or vaginal administration may be presented as a suppository or pessary with a suitable carrier. It is theorized that another method for reliable and sustained drug delivery can be obtained by the use of a vaginal ring containing a reservoir of the therapeutic compound to deliver a consistent daily or chronical release of hormone.

A microneedle array or intermittent injection of a slow release or micronized hormone preparation may find favor with the patient or prove quite efficacious in clinical practice.

The pharmaceutical composition may include one or more additives, depending on the pharmaceutically acceptable carrier, a preservative (including antioxidants), a dye, a binder, a suspending agent, thickeners, binders, buffers, a dispersing agent, a colorant, a disintegrate, an excipient, a diluent, a lubricant, a plasticizer, an oil or any combination of any of the foregoing. In particular embodiments, silica gel is used as a suspending agent. The amount of suspending agents will depend on the dosage and size of application, varying from about 0.01 g to about 1 gm. However, one skilled in the art will be able to best determine the amount of such additives. Examples of additional additives include, but are not limited to, sorbitol: talc; stearic acid; and dicalcium phosphate.

Suitable pharmaceutically acceptable additives include, but are not limited to, ethanol: water; glycerol; aloe vera gel; allantoin: glycerin: vitamin A and E oils; mineral oil; PPG2 myristyl propionate; vegetable oils and solketal.

Suitable binders include but are not limited to starch: gelatin: natural sugars, such as glucose, sucrose and lactose; corn sweeteners; natural and synthetic gums, such as acacia, tragacanth, vegetable gum, and sodium alginate: carboxymethylcellulose: polyethylene glycol: waxes; and the like.

Suitable lubricants include, but are not limited to, sodium oleate, sodium stearate, magnesium stearate, sodium acetate, and the like.

The composition may also include suitable preservatives, e.g., sodium benzoate, and other additives that may render the composition more suitable for application, e.g., sodium chloride, which affects the osmolarity of the preparation.

Suitable dispersing and suspending agents include, but are not limited to synthetic and natural gums, such as bentoite, vegetable gum, tragacanth, acacia, alginate, dextran, sodium carboxymethylcellulose, methylcellulose, polyvinyl-pyrrolidone and gelatin.

A suitable pharmaceutical diluent is, but is not limited to, water.

Additionally, various agents may be used to change the pH of the composition as necessary, including, for example, hydrochloric acid or sodium hydroxide, and antioxidants such as citric acid, ascorbic acid, fumaric acid and malic acid. Other possible antioxidants include palmitate, butylated hydroxyanisole, propylgallate, sodium ascorbate, and sodium metabisulfite. In particular embodiments, citric acid (0.1%) is used.

The mode of local administration and dosage forms will of course affect the therapeutic amounts of the agent [s] of the present invention which are desirable and efficacious for the given treatment application.

In summary this invention teaches the use of therapeutics and their methods of local delivery that can be used to treat pelvic floor disorders. Thus, the present invention provides sufficient clinical benefit on their own and can improve outcomes of non-surgical and surgical management.

### REFERENCES

The contents of these cited references are incorporated by reference herein in their entireties for all purposes.
1. Ashton Miller, JA and Delancey J O L. (2007) Functional Anatomy of the Female Pelvic Floor Ann. N.Y. Acad. Sci. 1101:266-296.
2. Versi E, Cardozo L D, Studd J W W, Brincat M, O'Dowd T M, Cooper D J., (1986) Internal urinary sphincter in maintenance of female continence. Br Med J 1986:292: 166-167.
3. Versi E. The significance of an open bladder neck in women, (1991) Br J Urol 1991; 68:42-3.
4. Emily S. Lukacz: Yahir Santiago-Lastra, Michael E. Albo. Linda Brubaker, 2017) Urinary Incontinence in Women A Review JAMA. 2017: 318 (16): 1592-1604.
5. Alavi K, Chan S, Wise P, Kaiser A M, Sudan R, Bordeianou L. (2015) Fecal Incontinence: Etiology. Diagnosis, and Management. J Gastrointest Surg. October; 19 (10): 1910-21.
6. AE, Rao S S C, Shin A S . . . (2017) Surgical Interventions and the Use of Device-Aided Therapy for the Treatment of Fecal Incontinence and Defecatory Disorders. Clin Gastroenterol Hepatol. December; 15 (12): 1844-1854. doi: 10.1016/j.cgh.2017.08.023.
7. Stanczyk F Z, Chaikittisilpa S, Sriprasert I, Rafatnia A, Mishell D R., (2019) Circulating androgen levels before and after oophorectomy in premenopausal and postmenopausal women. Climacteric. April; 22 (2): 169-174.
8. Labrie F, Martel C, Balser J., (2011) Wide distribution of the serum dehydroepiandrosterone and sex steroid levels in postmenopausal women: role of the ovary? Menopause: 18:30-43.
9. Gordon S E, Kraemer W J, Looney D P, Flanagan S D, Comstock B A, Hymer W C. (2014) The influence of age and exercise modality on growth hormone bioactivity in women. Growth Horm IGF Res. April-June; 24 (2-3): 95-103.
10. Russo E, Caretto M, Giannini A, Bitzer J, Cano A. Ceausu I, Chedraui P. Durmusoglu F, Erkkola R. Goulis D G, Kiesel L. Lambrinoudaki I, Hirschberg A L, Lopes P, Pines A, Rees M, van Trotsenburg M, Simoncini T., (2021) Management of urinary incontinence in postmenopausal women: An EMAS clinical guide. Maturitas. January; 143:223-230. doi: 10.1016/j.maturitas 09.005. Epub 2020 Sep. 30. PMID: 33008675.
11. Minassian V A, Bazi T, Stewart W F., (2017) Clinical epidemiological insights into urinary incontinence. Int Urogynecol J. 28 (5): 687-696.
12. Meyer I, Richter H E. Evidence-Based Update on Treatments of Fecal Incontinence in Women. (2016) Obstet Gynecol Clin North Am.; 43 (1): 93-119. doi: 10.1016/j.ogc.2015.10.005
13. Hendrix S L, Cochrane B B, Nygaard I E, Handa V L, Barnabei V M, Iglesia C, Aragaki A, Naughton M J, Wallace R B, McNeeley S G. (2005) Effects of estrogen with and without progestin on urinary incontinence. JAMA. February 23; 293 (8): 935-48.
14. Morales A, Buvat J, Gooren L J, et al. (2004) Endocrine aspects of sexual dysfunction in men. J Sex Med; 1:69-81.
15. Kumar N, Crozat A, Li F, Catterall J F, Bardin C W, Sundaram K (1999) 7alpha-methyl-19-nortestosterone, a synthetic androgen with high potency: structure-activity comparisons with other androgens. J Steroid Biochem Mol Biol 71:213-222.
16. Deslypere J P, Young M, Wilson J D, McPhaul M J (1992) Testosterone and 5 alpha-dihydrotestosterone interact differently with the androgen receptor to enhance transcription of the MMTV-CAT reporter gene. Mol Cell Endocrinol 88:15-22.
17. Lucas-Herald, Angela K et al. Genomic and non-genomic effects of androgens in the cardiovascular system: clinical implications. (2017) Clinical science (London, England:) vol. 131,13:1405-1418. doi: 10.1042/CS20170090.
18. Bach, F. L., Sairally, B., & Latthe, P. (2020). Effect of oestrogen therapy on faecal incontinence in postmenopausal women: a systematic review. International Urogynecology Journal, 31 (7), 1289-1297. https://doi.org/10.1007/s00192-020-04252-1.

1. A method of treatment of stress urinary incontinence, fecal incontinence, or both comprising administering at least one androgen to a human subject by local delivery, wherein said androgen is administered to the pelvic floor of the human subject or to areas having contact with the pelvic floor of the human subject or both to treat stress urinary incontinence, fecal incontinence, or both.

2. The method of treatment of claim 1, wherein the androgen is selected from the group consisting of dehydroepiandrosterone (DHEA), androstenedione (A), testosterone (T), and dihydrotestosterone (DHT).

3. The method of treatment of claim 1 that is treatment of stress urinary incontinence.

4. The method of treatment of claim 1, wherein the local delivery is selected from the group consisting of insertion into the vagina, into or through the vagina wall, into or through the anal canal wall, into the rectum, into or through the rectal wall, onto, into or through the perineum, or any combination thereof.

5. The method of claim 1, wherein the human subject is a male or female.

6. The method of claim 1, wherein the human subject is female.

7. The method of claim 1, wherein the therapeutic compound is in a delivery system for trans-mucosal delivery and in the form of a ring, pessary, gel, cream, ointment, suppository, or a microarray needle system.

8. The method of claim 1, wherein the at least one therapeutic compound is in a delivery system for absorption through the vaginal wall and in the form of a vaginal ring, vaginal pessary, gel, cream, ointment, vaginal suppository, or a microarray needle system.

9. The method of claim 1, wherein the therapeutic compound is delivered by injection into the vagina, into or through the vagina wall, into or through the anal canal wall, into the rectum, into or through the rectal wall, onto, into, or through the perineum, or any combination thereof.

10-11. (canceled)

12. The method of treatment of claim 1, wherein the androgen is dihydrotestosterone (DHT).

13. The method of treatment of claim 1, comprising local delivery of a combination of at least one androgen and ghrelin or a ghrelin analog.

14. The method of treatment of claim 1, wherein the local delivery of the at least one androgen minimizes potential negative effects of systemic exposure and reaches pelvic tissues before entering into systemic circulation thereby lowering plasma concentrations relative to pelvic tissue concentrations.

15. (canceled)

17. The method of treatment of claim 1 that is treatment of fecal incontinence.

18-23. (canceled)

24. The method of treatment of claim 1, wherein the at least one androgen is testosterone (T).

25. The method of treatment of claim 1, wherein the at least one androgen is androstenedione (A).

26. The method of treatment of claim 24, comprising local delivery of a combination of testosterone and ghrelin or a ghrelin analog.

27-30. (canceled)

31. The method of treatment of claim 1, wherein the at least one androgen is dehydroepiandrosterone (DHEA).

32. The method of treatment of claim 1 comprising local delivery of a combination of dihydrotestosterone (DHT) and ghrelin or a ghrelin analog.

33. (canceled)

34. The method of treatment of claim 1, wherein the at least one androgen is present in amounts ranging from about 0.2 mg per dose to about 200 mg per dose.

35. A method of treatment of stress urinary incontinence, fecal incontinence, or both comprising administering at least one ghrelin and/or ghrelin analogue to a human subject by local delivery, wherein said ghrelin and/or ghrelin analogue is administered to the pelvic floor of the human subject or to areas having contact with the pelvic floor of the human subject or both to treat stress urinary incontinence, fecal incontinence, or both.
